(19) United States
(12) Patent Application Publication
(10) Pub. No.: US 2024/0139458 A1
(43) Pub. Date: May 2, 2024

(54) TRACHEAL INTUBATION DEVICE FOR DELIVERY OF APNEIC OXYGENATION AND SUCTION

(71) Applicant: THE HOSPITAL FOR SICK CHILDERN, TORONTO (CA)

(72) Inventors: DIANE SOARES, MISSISSAUGA (CA); MARK OLIVER TESSARO, TORONTO (CA); CENGIZ KARSLI, TORONTO (CA); PETER ALEXANDER GORDON, TORONTO (CA)

(21) Appl. No.: 18/386,749

(22) Filed: Nov. 3, 2023

Related U.S. Application Data
(63) Continuation of application No. 16/608,251, filed on Oct. 25, 2019, now abandoned.
(60) Provisional application No. 62/489,624, filed on Apr. 25, 2017.

(51) Int. Cl. A61M 16/08; A61M 16/04; A61M 16/10; A61M 16/20

(52) U.S. Cl. CPC A61M 16/0816; A61M 16/0434; A61M 16/0463; A61M 16/1005; A61M 16/209

(57) ABSTRACT

This disclosure discloses a tracheal intubation device designed to improve safety during intubation attempts. The device allows for the delivery of medical gas (such as oxygen) flow through the endotracheal tube itself during intubation attempts. This moves the source of apneic oxygen lower in the patient's airway to bypass anatomy that otherwise can obstruct medical gas flow. The device can also switch on-demand to generating suction through the endotracheal tube to clear away fluids that block the intubator's view and that block medical gas flow. There are currently no devices that can deliver both medical gas and on-demand suction using the endotracheal tube as the flow conduit during the process of intubation. The present device accomplishes these tasks while incorporating non-obvious elements to optimize its use and efficacy.

### FIELD OF THE DISCLOSURE

This disclosure relates to devices for the delivery of apneic oxygenation and suction, and more specifically devices for delivery of apneic oxygenation and on-demand suction through an endotracheal tube during the process of intubation.

### BACKGROUND

Airway intubation is the placement of an endotracheal tube into a patient's airway to a depth below the vocal cords. It is a procedure that can occur electively in order to protect the airway and respiration of a patient during surgery or can occur in a critical illness. It is a high-risk procedure as it occurs when patients are apneic (not breathing) either due to their illness or from medications, and a narrow time window is available to the intubator prior to a dangerous decrease in the patient's blood oxygen levels, which significantly are worsened in the presence of disease. An intubation attempt may be prolonged due to airway anatomy that either prevents good visualization of the airway or due to difficulty advancing the endotracheal tube. It may also be prolonged if fluids (secretions or blood) obscure the intubator's view of the endotracheal tube's path, requiring a suction catheter to be introduced into the patient's mouth.

Apneic oxygenation is the delivery of flowing oxygen into a patient's nares via nasal cannula tubing during intubation attempts, and provides a proven safety benefit by delaying the time to oxygen desaturation. This oxygen flow can be blocked by the same airway conditions or fluids mentioned above, negating this safety benefit. Therefore, providing a tracheal intubation device that can, while being inserted into the patient's airway, be switched by the clinician from delivering oxygen and providing suction if needed would be very advantageous and increase the safety of the insertion procedure.

### SUMMARY

Provided is a tracheal intubation device designed to improve safety during intubation attempts. The device allows for the delivery of oxygen (or other medical gas) flow through the endotracheal tube (or through a laryngeal mask airway or other supraglottic device that secures a patient's airway) itself during intubation attempts. This moves the source of apneic oxygen lower in the patient's airway to bypass nasopharynx anatomy that otherwise can obstruct oxygen flow. The device can also switch on-demand to generating suction through the endotracheal tube to clear away fluids that block the intubator's view and that block oxygen flow. There are currently no devices that can deliver both oxygen and on-demand suction using the endotracheal tube as the flow conduit during the process of intubation. The present device accomplishes these tasks while incorporating non-obvious elements to optimize its use and efficacy.

The present device comprises an endotracheal tube adaptor that connects to the patient-exterior-end of the endotracheal tube, a control unit that connects to standard medical gas and suction tubing and controls medical gas and suction delivery, and a length of lightweight flexible tubing that connects the adaptor to the control unit.

During intubation, a semi-rigid stylet is placed within the endotracheal tube to make it rigid enough to allow it to advance rather than bending as it slides along airway surfaces. The distal end of this stylet sits at the airway-end of the endotracheal tube, while the proximal end of the stylet emerges from the patient-exterior-end of the endotracheal tube, and prevents the application of oxygen supply tubing to the endotracheal tube. Our device's adaptor to the patient-exterior-end of the endotracheal tube allows for a stylet using an in-line self-sealing minimal-leak port, allowing oxygen flow or suction to occur via a second off-axis adaptor conduit.

An intubation requires the clinician to use one hand to control a laryngoscope (a blade with a light source) to generate a view of the airway, and the other hand to manipulate the endotracheal tube towards the vocal cords. Our device utilizes lightweight flexible tubing to connect its endotracheal tube adaptor to a control unit. The on-demand suction control components as well as bulky tubing running from medical gas and suction sources are thus kept away from the patient-exterior-end of the endotracheal tube. This maintains a clear visual axis for the intubator, and ensures that the endotracheal tube does not become difficult to manipulate, as it would if the mass of these components were added directly to its patient-exterior-end. It allows an assistant to activate on-demand suction at a location where their manipulation of the device will not obscure the intubator's view or create traction on the endotracheal tube that would make it more difficult to manipulate.

The application of high pressures into a patient's airway can cause airway tissue damage (barotrauma). The present control device contains a pressure-release valve that activates automatically if pressure within the flexible tubing rises above a set level, thus preventing barotrauma.

Thus disclosed herein is an airway device for delivering medical gas to a patient, comprising:
an airway access device configured to be coupled to a patient's airway;
an airway access device adaptor connected to and in flow communication with the airway access device;
a medical gas input control unit including a housing having first and second ends, the second end of said control united being connected to the airway access device adapter, the control unit including a medical gas supply coupling located at the first end of the housing connectable to a source of medical gas, a pressure limiting device located downstream of the medical gas supply coupling, and wherein the pressure limiting device is configured to limit medical gas flow upon a pressure of medical gas in the pressure limiting device exceeding a preselected threshold pressure; and
the airway access device adapter is configured to form a gas tight seal with the airway access device when coupled to said airway access device.

The device medical gas input control unit may further comprise a suction supply coupling located at the first end of the housing and being connectable to a source of suction, and the control unit may include a hand-operated control mechanism for controlling both suction and medical gas flow.

The hand-operated control mechanism may be a finger activated switch for switching between the source of suction and the source of medical gas, and the control unit may be configured such that when the finger activated mechanism is not activated, medical gas flows to the airway access device, and when activated the medical gas flow is stopped and suction is applied to the airway access device, so that medical gas and suction cannot be provided at the same time.

The hand-operated control mechanism may be a finger activated switch for switching between the source of suction and the source of medical gas and the control unit may be configured such that when the finger activated mechanism is not activated, medical gas flows to the airway access device, and when activated the medical gas flow is reduced to a preselected flow rate and suction is simultaneously applied to the airway access device, so that medical gas and suction are provided at the same time.

The airway access device adaptor may be connected to, and in flow communication with, the airway access device by means of elongate flexible tubing.

The pressure limiting device may be any one of a pressure relief valve, a pressure regulating valve, a rupture disc, an aperture, or a breather vent.

The airway access device may be any one of an endotracheal tube, Laryngeal mask airway, tracheostomy tube, nasopharyngeal airway or tube, oropharyngeal tube, cricothyrotomy tube, and the like.

The present disclosure provides an airway device for delivering medical gas to a patient, comprising:
an airway access device configured to be coupled to a patient's airway;
an airway access device adaptor connected to and in flow communication with the airway access device;
a medical gas input control unit including a housing having first and second ends, the second end of said control united being connected to said airway access device adapter, the control unit including a medical gas supply coupling located at said first end of the housing connectable to a source of medical gas, a pressure limiting device located downstream of the medical gas supply coupling, and wherein the pressure limiting device is configured to limit medical gas flow upon a pressure of medical gas in the pressure limiting device exceeding a preselected threshold pressure;
a suction supply coupling located at the first end of the housing and being connectable to a source of suction, the control unit including a hand-operated control mechanism for controlling both suction and medical gas flow; and
the airway access device adapter being configured to form a gas tight seal with the airway access device when coupled to the airway access device.

The hand-operated control mechanism may be a finger activated switch for switching between the source of suction and the source of medical gas, the control unit being configured such that when the finger activated mechanism is not activated, medical gas flows to the airway access device, and when activated the medical gas flow is stopped and suction is applied to the airway access device, so that medical gas and suction cannot be provided at the same time.

The hand-operated control mechanism may be a finger activated switch for switching between the source of suction and the source of medical gas, the control unit being configured such that when the finger activated mechanism is not activated, medical gas flows to the airway access device, and when activated the medical gas flow is reduced to a preselected flow rate and suction is simultaneously applied to the airway access device, so that medical gas and suction are provided at the same time.

The airway access device adaptor may be connected to, and in flow communication with, the airway access device by means of elongate flexible tubing.

The pressure limiting device may be any one of a pressure relief valve, a pressure regulating valve, a rupture disc, an aperture, or a breather vent.

The airway access device may be any one of an endotracheal tube, Laryngeal mask airway, tracheostomy tube, nasopharyngeal airway or tube, oropharyngeal tube, cricothyrotomy tube, and the like.

The present disclosure provides an airway device for delivering medical gas to a patient, comprising:
an endotracheal tube having a first end configured to be inserted into a patient's airway and an opposed second end;
an endotracheal tube adaptor attached to the second end configured to have one end of an elongate flexible tube attached thereto; and
a hand operated medical gas input control unit including a housing having first and second opposed ends, a second end of the elongate flexible tube being connected to the second end of said housing, the control unit including a medical gas supply coupling connectable to a source of medical gas and a pressure limiting device located downstream of the medical gas supply coupling between said medical gas supply coupling and the first end of the housing, wherein the pressure limiting device is configured to vent the medical gas out of the housing upon a pressure of medical gas in the flexible tube exceeding a preselected threshold pressure and
the endotracheal tube adapter configured to form a gas tight seal with said endotracheal tube when coupled to said airway access device.

The medical gas input control unit may further comprise a suction supply coupling located at the first end of the housing and being connectable to a source of suction, the control unit including a hand-operated control mechanism for controlling both suction and medical gas flow.

The pressure limiting device may be a pressure relief valve is located adjacent to the second end of the housing with the medical gas supply coupling being located between said pressure relief valve and said suction supply coupling.

The suction supply coupling may be located adjacent to the first end of the housing, and wherein the medical gas supply coupling is located adjacent to the second opposed end, and wherein said pressure limiting device is a pressure relief valve located between the medical gas supply coupling and the suction supply coupling.

A further understanding of the functional and advantageous aspects of the disclosure can be realized by reference to the following detailed description and drawings.

### BRIEF DESCRIPTION OF THE DRAWINGS

The disclosure will be more fully understood from the following detailed description thereof taken in connection with the accompanying drawings, which form part of this application, and in which:

FIG. 1 is a front view of an embodiment of the tracheal intubation device showing the airway unit, endotracheal tube, tubing component, and control unit;

FIG. 2 is a perspective view of the device of FIG. 1;

FIG. 3 is a perspective view of the control unit of the device of FIG. 1;

FIG. 4A is a top-down view of the control unit of FIG. 3 with a cutting plane;

FIG. 4B is a section view of the control unit taken along the plane A-A of FIG. 4A;

FIG. 5 is a wire-frame view of the control unit of FIG. 3 showing gas streamlines when on-demand suction is activated;

FIG. 6 is a front view of an embodiment of the tracheal intubation device where the tracheal intubation device is for medical gas delivery only;

FIG. 7 is a diagram of an alternate embodiment of the control unit;

FIG. 8 is a diagram of an alternate embodiment of the control unit having separate medical gas and suction chambers;

FIG. 9 is a diagram of an alternate embodiment of the control unit having separate medical gas and suction sections;

FIG. 10 is a top-down view of an alternate embodiment of the tracheal intubation device with the endotracheal tube adaptor and tubing component of the device of FIG. 1 and the control unit of FIG. 7;

FIG. 11 is view of the device of FIG. 10 being used with a mannequin showing the airway unit, a tubing component, and a control unit;

FIG. 12 is a view of the device of FIG. 10 being used with a mannequin while suction is active;

FIG. 13A is a diagram of an alternate embodiment of the tracheal intubation device as a portable unit with stand-alone medical gas and suction supplies;

FIG. 13B is a perspective view of the tracheal intubation device of FIG. 13A;

FIG. 14A is an isometric view of another embodiment of a hand operated control unit for the tracheal intubation device disclosed herein;

FIG. 14B is a disassembled view of the handheld unit of FIG. 14A;

FIG. 15A is a rear view of the handheld unit of FIG. 14A looking along arrow 15A of FIG. 14A;

FIG. 15B is a bottom view of the handheld unit of FIG. 14A looking along arrow 15B of FIG. 14A;

FIG. 16A is a side view of a valve forming part of the hand held control unit of FIG. 14A;

FIG. 16B is a view of the valve of FIG. 16A but the input and output gas and suction connectors coupled thereto;

FIG. 17A is an isometric view of a partially disassembled control unit of FIG. 14A;

FIG. 17B is a side elevation view of the control unit of FIG. 14A;

FIG. 18A is an isometric view of the hand operate control unit, showing the interior structure of the medical gas and suction flow pathways in its default state with the finger operated control button unengaged by the clinician such that in this default stated medical gas is flowing into the patient's airway;

FIG. 18B is an isometric view of the control unit similar to FIG. 18A showing suction and oxygen flow pathways through the control unit but now with the control button depressed or activated by the clinician so that the medical gas is vented to atmosphere and the suction is engaged to clear the patient's airway;

FIG. 19 is an isometric view of the assembled tracheal intubation device using the hand operated control unit of FIG. 14A;

FIG. 20A is a side view of a gas and suction coupling connecting the hand operated control unit of FIG. 14A to the tracheal intubation tube with the coupled to the adapter attached to the intubation tube;

FIG. 20B is a perspective view of the coupling of FIG. 20A connected to the tracheal intubation tube and tubes for delivering medical gas and suction from the hand operated unit of FIG. 14A to the tracheal intubation tube;

FIG. 21A is an isotropic view showing the sealing cap forming part of an airway access device adaptor forming part of the present device; and

FIG. 21B is a cross-sectional view of the sealing cap of FIG. 21A.

### DETAILED DESCRIPTION

The devices described herein are directed, in general, to tracheal intubation devices and more specifically to tracheal intubation devices for delivery of apneic oxygenation (or other medical gases) and on-demand suction. Although embodiments of the present invention are disclosed herein, the disclosed embodiments are merely exemplary and it should be understood that the invention relates to many alternative forms, including different shapes and sizes. Furthermore, the Figures are not drawn to scale and some features may be exaggerated or minimized to show details of particular features while related elements may have been eliminated to prevent obscuring novel aspects. Therefore, specific structural and functional details disclosed herein are not to be interpreted as limiting but merely as a basis for the claims and as a representative basis for enabling someone skilled in the art to employ the present invention in a variety of manners.

As used herein, the terms “comprises”, “comprising”, “includes” and “including” are to be construed as being inclusive and open ended, and not exclusive. Specifically, when used in this specification including claims, the terms “comprises”, “comprising”, “includes” and “including” and variations thereof mean the specified features, steps or components are included. These terms are not to be interpreted to exclude the presence of other features, steps or components.

As used herein, the terms “about” and “approximately”, when used in conjunction with ranges of dimensions, compositions of mixtures or other physical properties or characteristics, is meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present invention.

As used herein, the coordinating conjunction “and/or” is meant to be a selection between a logical disjunction and a logical conjunction of the adjacent words, phrases, or clauses. Specifically, the phrase “X and/or Y” is meant to be interpreted as “one or both of X and Y” wherein X and Y are any word, phrase, or clause.

As used herein the phrase “airway access device” refers to any medical device used by a clinician to access a patient's airway. Thus the airway access device may include any of, but is not limited to, an endotracheal tube, Laryngeal mask airway, tracheostomy tube, nasopharyngeal airway or tube, oropharyngeal tube, cricothyrotomy tube, and the like.

While the following disclosure and figures illustrates the present airway device for delivering medical gas and suction to a patient using an endotracheal tube as the airway access device, it will be appreciated that with minor design modifications the present device can be adapted for any airway access device, such as, but not limited, to those mentioned above.

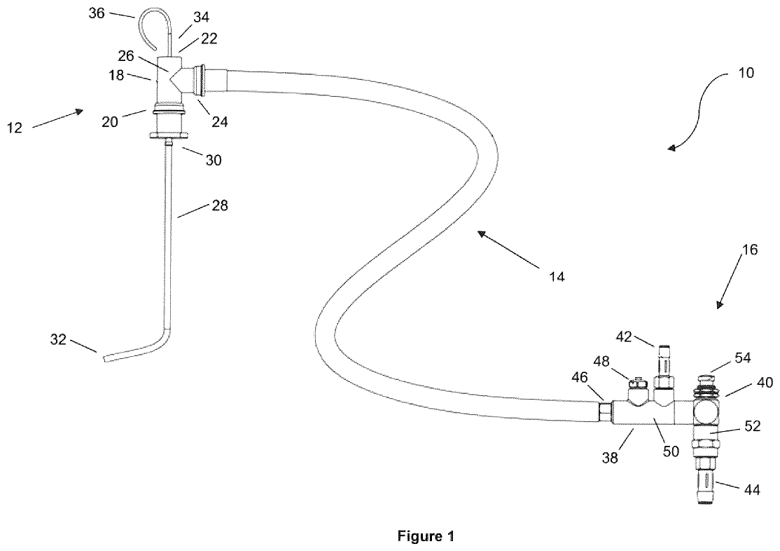

The tracheal intubation device of the present disclosure, an embodiment of which is shown in FIG. 1 at 10 generally comprises an endotracheal tube adaptor 12, a tubing component 14 and a control unit 16.

The endotracheal tube adaptor 12 of the present disclosure, an embodiment of which is shown in FIG. 1, generally comprises a body 18, an airway connector 20 attached to the body 18, a stylet accommodator 22 attached to the body 18 and a tubing port 24 attached to the body 18. The body 18 has a hollow chamber 26 made of rigid material. In a preferred embodiment of the disclosure, the body 18 is cylindrical.

The airway connector 20 is shaped such that the patient-exterior end 30 of an endotracheal tube 28 can be removably attached to the airway connector 20 such that medical gas and/or suction may be conducted through the airway connector between the airway end 32 of the endotracheal tube 28 and the body chamber 26. In a preferred embodiment, the airway connector 20 is cylindrical and is shaped such that the universal 15 mm diameter connector of an endotracheal tube or laryngeal mask airway or other supraglottic airway device can be removably attached by snug fit to the airway connector 20. In the embodiment of the disclosure shown in FIG. 1, the airway connector is shaped such that the patient-exterior-end 30 of the endotracheal tube 28 is secured by snug fit within the airway connector.

The stylet accommodator 22 is attached to the body 18 of the endotracheal tube adaptor 12 and allows a semi-rigid stylet 34 to be removably positioned within the endotracheal tube 28 during intubation to make the endotracheal tube 28 rigid enough to allow it to advance into the airway rather than bending on airway surfaces. The diameter of the endotracheal tube 28 is greater than the diameter of the stylet 34 allowing medical gas or suction to be delivered during intubation. The stylet accommodator 22 is a self-sealing minimal-leak port, allowing medical gas and suction flow between the tubing component 14 and the endotracheal tube 28 via the endotracheal tube adaptor 12.

In the embodiment of the disclosure shown in FIG. 1, the stylet accommodator 22 is an enclosure that encloses the distal end 36 of the stylet 34 such that the stylet 34 can be inserted into the endotracheal tube 28 during intubation.

In an alternate embodiment of the disclosure, the stylet accommodator 22 is a removable plastic cap with a hole in the center of said cap so an intubation stylet can be inserted into the endotracheal tube through the hole in said cap. It will be appreciated by one skilled in the art that a cap with no hole in it can be used if the device of the present disclosure is being used without an intubation stylet.

The tubing port 24 is attached to the body 18 of the endotracheal tube adaptor 12 and is shaped such that the tubing component 14 can is removably attachable to the endotracheal tube adaptor 12. The tubing port 24 conducts medical gas flow or suction between the body chamber 26 of the endotracheal tube adaptor 12 and the tubing component 14. In a preferred embodiment shown in FIG. 1, the tubing port 24 is a hollow, open-ended cylinder. The tubing component 14 is attachable to the tubing port 24 either by adhesive, a snug fit, a crimping or clamping mechanism, a banding mechanism, a riveting mechanism and/or a flange mechanism. The tubing port 24 is either shaped such that the tubing component fits around the tubing port or the tubing component fits within the patient side tubing attachment.

In the embodiment of the present disclosure shown in FIG. 1, the airway connector 20 is attached to one end of the body 18 and the stylet accommodator 22 is attached to the other end of the body 18. Furthermore, the stylet accommodator 22 is in line with the central axis of the airway connector 20. The tubing port 24 is attached to the side face of the body 18 where the central axis of the tubing port is at an angle to the central axis of the airway connector 20.

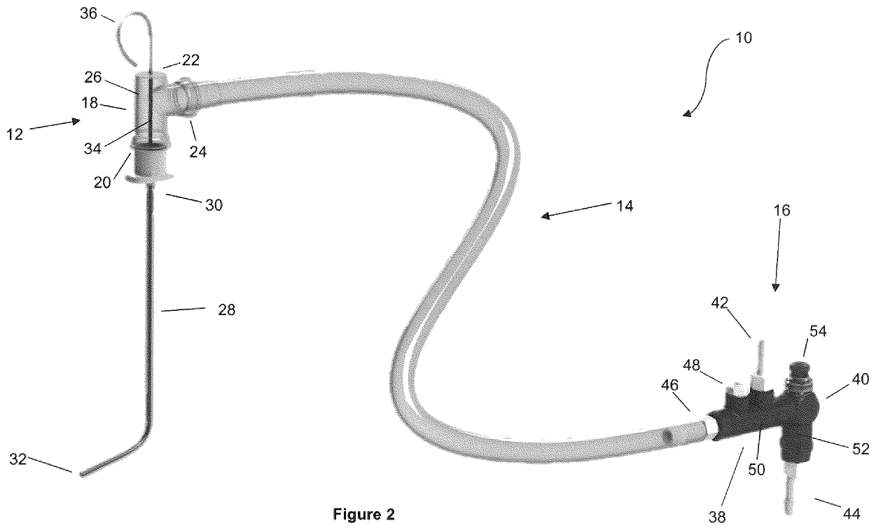

The tubing component 14, an embodiment of which is shown in FIG. 1, consisting of semi-rigid or flexible, lightweight tubing, attaches at one end to the tubing port 24 of the endotracheal tube adaptor 12 and attaches at the other end to a tubing component port 46 of the control unit 16. The tubing component 14 conducts medical gas flow or suction between the endotracheal tube adaptor 12 and the control unit. The flexible nature of the tubing component 14 of the tracheal intubation device 10 is shown in FIG. 2.

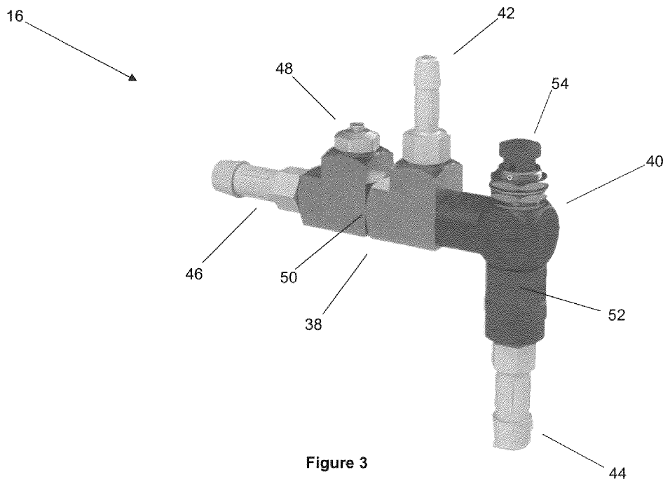

The control unit 16, shown in FIG. 3, generally comprises a body 38, a control apparatus 40, a medical gas input port 42, a suction port 44, a tubing component port 46 and a pressure release valve 48.

The body 38 of the control unit 16 has a hollow main chamber 50 made of rigid material. In a preferred embodiment, the body 38 is a cylinder.

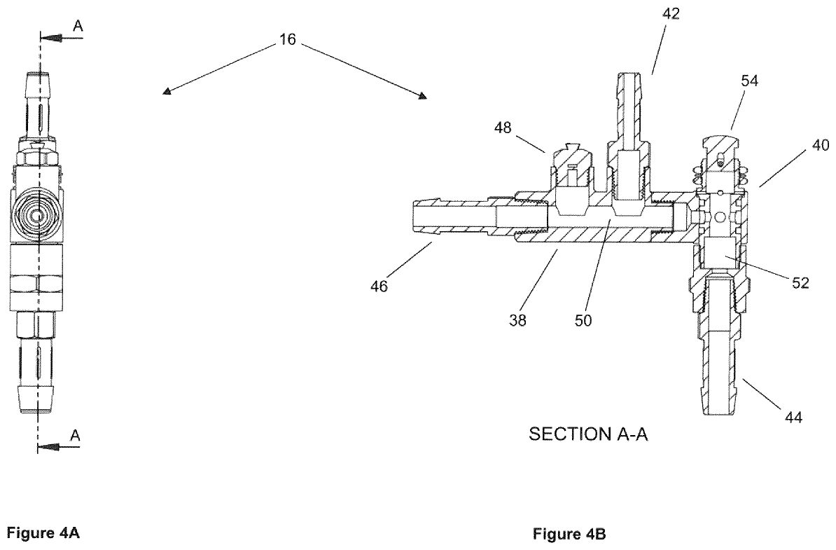

In the embodiment of the present disclosure shown in FIG. 3, the tubing component port 46 is attached to one end of the body 38 and the suction port 44 is attached to the other end of the body 38. The main chamber 50, best shown in FIG. 4B is between the tubing component port 46 and the control apparatus 40. The medical gas input port 42 is attached to the side of the body 38 and the pressure release valve 48 is attached to the side of the body 38 between the medical gas input port 42 and the tubing component port 46.

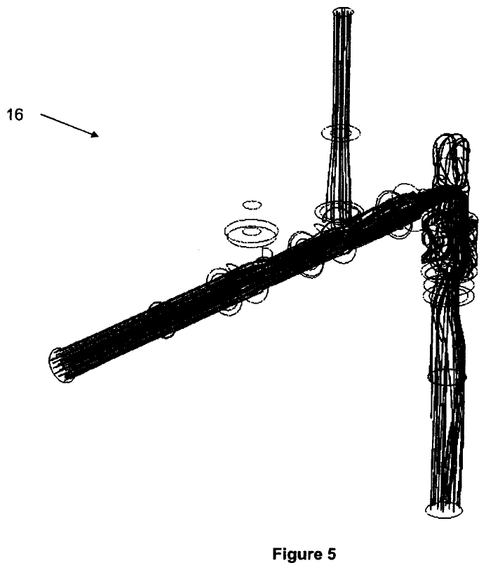

The control apparatus 40 enables an operator to switch from medical gas supply to on-demand suction. In the embodiment shown in FIG. 3, the suction port 44 is separated from the main chamber 50 by the control apparatus 40. The suction chamber 52, best shown in FIG. 4B is the space between the control apparatus 40 and the suction port 44. The medical gas input port 42, tubing component port 46, and pressure release valve are all attached to the body 38 of the control unit. The control apparatus 40 is attached such that when it is not activated, the suction chamber 52 is separated from the main chamber 50. When the control apparatus 40 is not activated, medical gas is delivered through the medical gas input port into the main chamber 50 and through the tubing component port 46 into the tubing component 14. The medical gas then moves from the tubing component 14 through the tubing attachment 24 into the body chamber 26 of the endotracheal tube adaptor 12 and from the body 18 through the airway connector 20 into the endotracheal tube 28 and the medical gas flows through the endotracheal tube 28 and out of the airway end 32 of the endotracheal tube 28. The control apparatus 40 is attached such that when it is in the activated position, a channel opens between the suction chamber 52 and the main chamber 50, allowing for a suction force to be conducted through the tracheal intubation device 10. When the control apparatus 40 is in the activated position the suction force is sufficient to clear blockages that occur during intubation. Any fluids and semi-solids (such as mucous) preventing medical gas flow through the endotracheal tube or obscuring the incubator's view can be extracted when suction is activated. When suction is activated, matter is sucked into the airway end 32 of the endotracheal tube 28 and moves through the endotracheal tube into the body 18 of the endotracheal tube adaptor 12 through the airway connector 20. From the body 18 the matter moves through the tubing attachment 24 into the tubing component 14. From the tubing component 14 the matter moves through the tubing component port 46 into the main chamber 50 of the control unit 16. When suction is activated medical gas flow continues from the medical gas source (not shown) into the main chamber 50 with any matter extracted from the patient through the endotracheal tube adaptor 12 and tubing component 14, as shown in FIG. 5. From the main chamber 50, the medical gas and extracted matter move through the activated control apparatus 40 and through the suction port 44 into the suction tubing. In the embodiment of the control unit 16 shown in FIGS. 1 through 4, the control unit is constructed out of off the shelf components.

In the embodiment of the disclosure shown in FIG. 1, the control apparatus 40 is an off-the shelf valve with the default state of the valve being closed. The control valve has an automatic return mechanism such that the valve is closed when no force is being applied to the button 54. When a sufficient pushing force is applied to the button 54, the control valve 40 opens and when said force ceases to act on the button 54, the control valve 40 returns to the closed position.

The medical gas input port 42 attaches to the body 38 of the control unit 16 such that a medical gas supply tube is attachable to the medical gas input port 42 and medical gas is able to flow from a medical gas supply tube through the medical gas input port 42 into the main chamber 50. In the embodiment shown in FIG. 3, the medical gas input port is a rigid hollow tapering cone with external ridges to secure a medical gas supply tube around the rigid hollow tapering cone. The rigid hollow tapering cone is used as a male connector component for standard medical gas tubing.

The suction port 44 attaches to the body 38 of the control unit 16 such that a suction supply tube is attachable to the suction port and suction is able to be supplied to the suction chamber 52 of the control unit 16. The suction port enables the flow of matter and medical gas from the suction chamber 52 through the suction port 44 into the suction supply tube if the control apparatus 54 is activated. In the embodiment shown in FIG. 3, the suction port is a rigid hollow tapering cone with external ridges to secure a suction supply tube around the rigid hollow tapering cone. The rigid hollow tapering cone is used as a male connector component for standard medical suction tubing.

The tubing component port 46 is attached to the body 38 such that the end of the tubing component 14 that is not attached to the endotracheal tube adaptor 12 is removably attached to the tubing component port 46. The tubing component port 46 enables medical gas flow or suction to be conducted between the main chamber 50 of the control unit 16 and the tubing component 14. In the embodiment shown in FIG. 3, the tubing component port 46 is a rigid hollow tapering cone with external ridges to secure the tubing component 14 around the rigid hollow tapering cone.

The pressure release valve 48 is attached to the body 38 such that the valve opens if the pressure within the main chamber 50 is above a predetermined level. When the pressure release valve 48 is opened, any pressure throughout the tracheal intubation device 10 that is above the setting on the pressure release valve 48 is released. The pressure release valve 48 opens to release pressure within the tracheal intubation device 10 if the pressure within the system is at a dangerous level, decreasing the risk of barotrauma (tissue damage in the airway due to high pressure). It will be appreciated by those skilled in the art that a cap may be placed around the pressure release valve 48 to disable the pressure release valve 48. By setting the flow of medical gas into the device at a level that leads to opening of the pressure release valve 48, the device will also provide continuous positive airway pressure at a level determined by the setting of the pressure release valve 48. This pressure release valve 48 may have a single pressure level at which it opens, or may be of an adjustable design to allow it to open at any selected pressure level within a range of pressures, 1 cm H₂0 to 100 cm H₂0, for example.

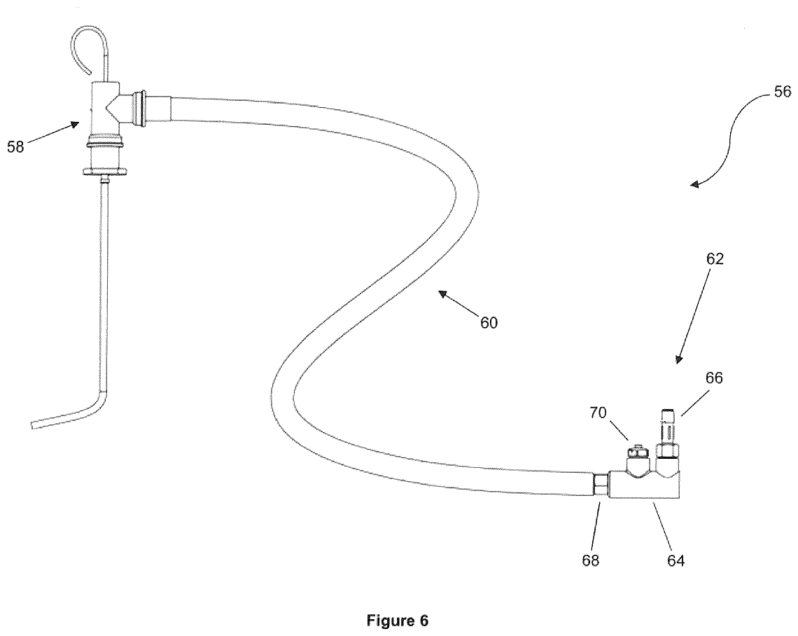

In an alternate embodiment of the endotracheal intubation device shown in FIG. 6, there are no suction components, and the device is used only for the delivery of medical gas flow. The endotracheal intubation device 56 consists of an endotracheal tube adaptor 58, a tubing component 60 and a control unit 62. The endotracheal tube adaptor 58 and the tubing component 60 of device 56 being the same as endotracheal tube adaptor 12 and tubing component 14 of the device 10 of FIG. 1. Control unit 62 is similar to control unit 16, however control unit 62 consists of a body 64, a medical gas input port 66, a tubing component port 68 and a pressure release valve 70, and does not have any suction components or a control apparatus.

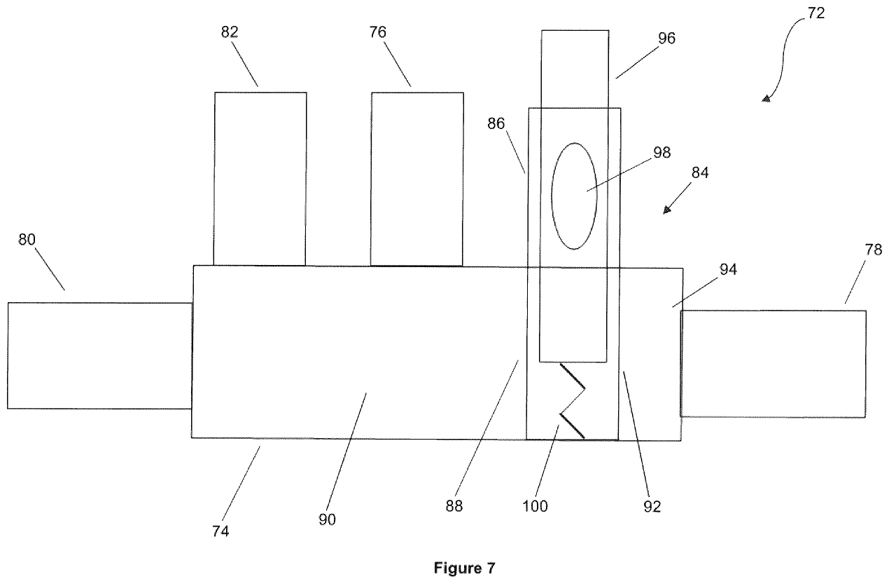

In FIG. 7 an alternate embodiment of the control unit is shown. This embodiment 72 consists of similar components to control unit 16, the components being a body 74, a medical gas input port 76, a suction port 78, a tubing component port 80 and a pressure release valve 82. However, the control apparatus 84 of control unit 72 is a barrel and plunger mechanism. The barrel and plunger mechanism comprises a barrel 86 that is attached to the body 74 and has an opening 88 to the main chamber 90 of the control unit 72 and there is another opening 92 to the suction chamber 94 of the control unit 72. The plunger 96 has a channel 98 running through it and the plunger is positioned within the barrel 86 with a spring 100 in one end of the barrel 86 between the barrel 86 and the plunger 96. The plunger 96 is positioned within the barrel 86 such that the plunger cannot slide out of the barrel 86 and the spring 100 exerts a force on the plunger such that the default state of the control apparatus 84 is not activated. When a pushing force is exerted on the end of the plunger 96 without the spring 100, the plunger 96 translates within the barrel 86 such that the channel 98 aligns with the openings 88 and 92, giving the control apparatus 84 an activated state. If this force ceases to be exerted on the plunger 96 the spring 100 exerts a force on the plunger 96 translating the plunger 96 such that the channel 98 is not aligned with the channels 88 and 92, returning the control apparatus 84 to a not activated state.

It will be appreciated by those skilled in the art that any other elastic object with a similar function to the spring 100 may be used so that the apparatus automatically returns to a default state. For example, sponge can be used instead of a spring. Alternatively, it will also be appreciated by those skilled in the art that the control apparatus 84 of the control unit 72 does not need an automatic return mechanism if it is not desirable.

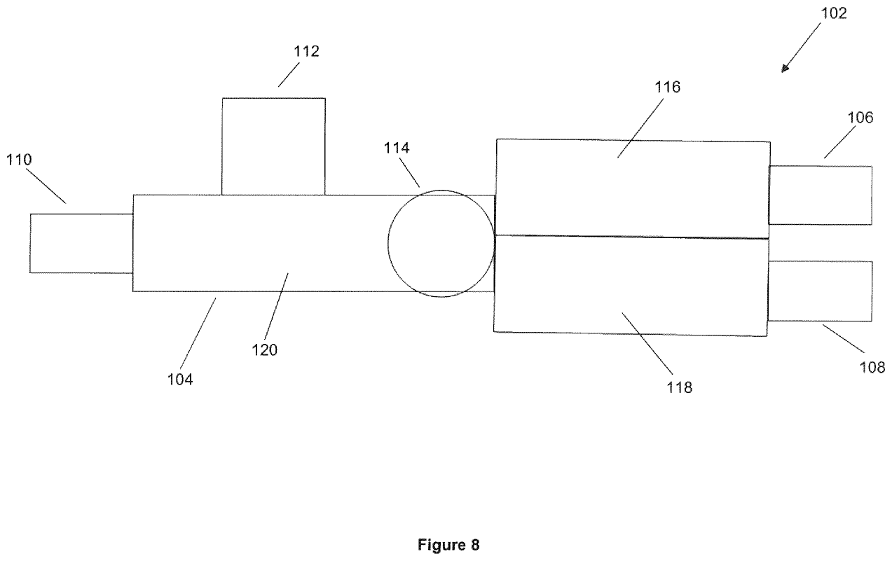

In FIG. 8 another type of control unit is shown at 102. This control unit 102 is comprised of similar components to control unit 16, the components being a body 104, a medical gas input port 106, a suction port 108, a tubing component port 110, a pressure release valve 112 and a control apparatus. The control apparatus of control unit 102 is a channel selector mechanism 114, such as but not limited to, a stopcock. The channel selector mechanism 114 allows either medical gas flow from the medical gas input port 106 through a medical gas chamber 116 or suction from the suction port 108 through a suction chamber 118 to communicate to the body chamber 120 of the control unit 102. In unit 102, when suction is engaged, gas flow is blocked from entering the body chamber 120 of the control unit 102. The channel selector mechanism 114 ideally possesses a default state in which medical gas communicates to the body chamber 120 of the control unit 120. Actively engaging the channel selector 114 allows suction to be communicated to the body chamber 120 while blocking medical gas flow. The channel selector 114 also ideally possesses an automatic return feature such that once the channel selector 114 is no longer actively engaged, the channel selector 114 returns to the default state in which medical gas is communicated to the body chamber 120 of the control unit 102. The blockage of medical gas flow during the activation of suction prevents the medical gas flow from reducing the total suction force flowing through the body chamber of the device. If medical gas continued to flow through the body chamber simultaneously, the effective suction force, and therefore the ability to remove material, would be reduced. In a separate embodiment, the medical gas could be limited to a preselected flow rate, instead of fully blocking, to achieve a similar result.

It will be appreciated by those skilled in the art that any other channel selection feature with a similar function to a stopcock may be used so that the control apparatus automatically returns to a default state. Alternatively, it will also be appreciated by those skilled in the art that the channel selector of the control unit does not need an automatic return mechanism if it is not desirable.

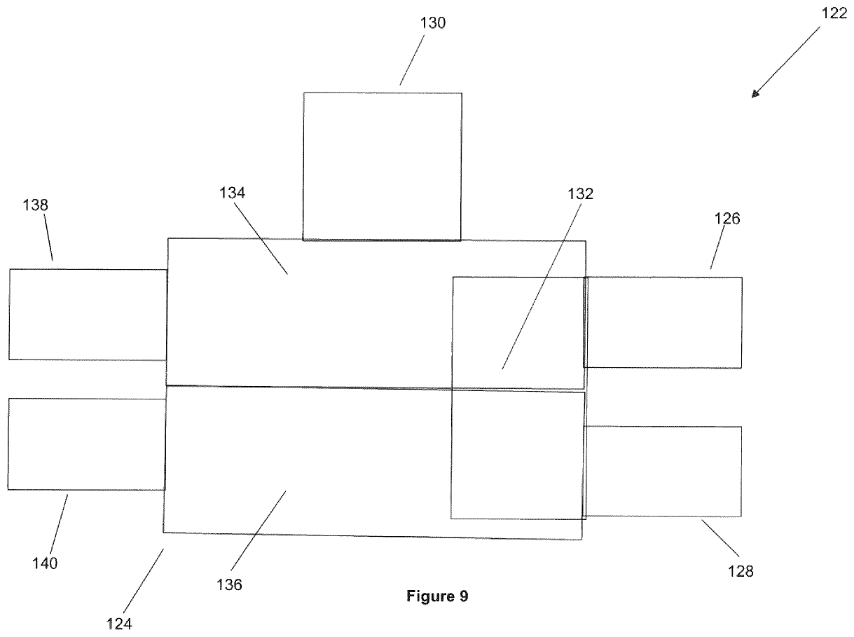

In FIG. 9 an alternate embodiment of the control unit is shown. This embodiment 122 is comprised of similar components to control unit in FIG. 8, the components being a body 124, a medical gas input port 126, a suction port 128, a tubing component port, a pressure release valve 130 and a channel selector mechanism 132. However, in this embodiment, the body 124 houses two separate channels, one for medical gas 134 and one for suction 136. As in FIG. 8, the channel selector mechanism 132 allows only medical gas or suction to be engaged, but not both at once. Similar to the mechanism shown in FIG. 8, the channel selector mechanism 132 shown in FIG. 9 ideally possesses a default state in which medical gas flow is engaged while suction is not engaged. When suction is engaged, gas flow is blocked from entering the medical gas channel 134 within the control unit 122. Actively engaging the channel selector 132 allows suction to be communicated to the suction channel 136 within the body 122 while blocking medical gas flow to the medical gas channel 134 within the body 122.

The channel selector 132 also preferably possesses an automatic return feature such that once the channel selector 132 is no longer actively engaged, the channel selector 132 returns to the default state in which medical gas flow is communicated to the medical gas channel 134 within the body and suction is blocked from communicating with the suction channel 136 within the body of the control unit. In this embodiment, the tubing component consists of two separate channels, one for medical gas flow 138 and one for suction 140, and the tubing attachment of the endotracheal tube adaptor is different from tubing attachment 24, such that it is compatible with the tubing component of the present embodiment. In the present embodiment, the medical gas tube and suction tube may be connected to form one dual channel tubing component. This dual channel tubing may consist of side-by-side channels, or one smaller diameter channel dwelling within one large diameter channel.

It will be appreciated by those skilled in the art that any other channel selection feature with a similar function to a stopcock may be used so that the apparatus automatically returns to a default state. Alternatively, it will also be appreciated by those skilled in the art that the channel selector of the control unit does not need an automatic return mechanism if it is not desirable.

In addition, it will be understood that the present tracheal intubation device may be produced without suction and the associated suction control, and instead provides only medical gas supply and its associated control unit.

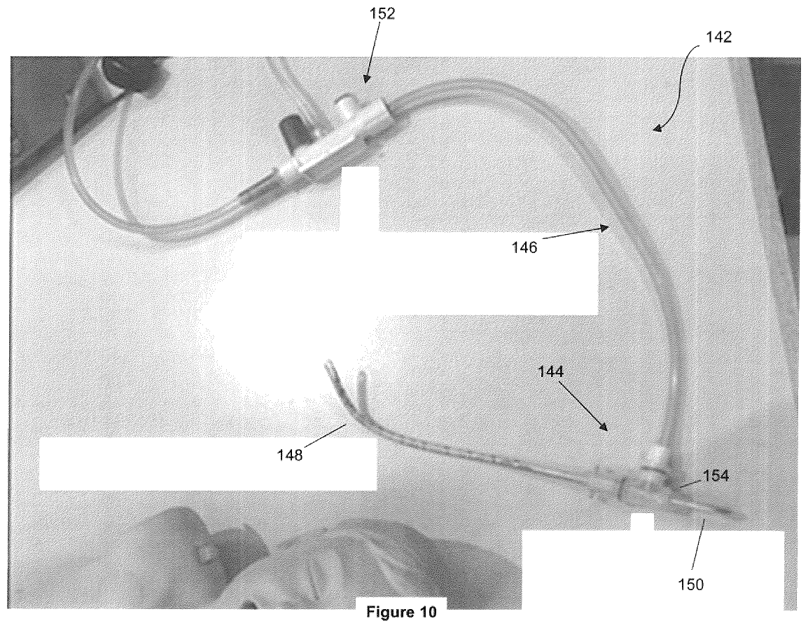

Another tracheal intubation device 142 is shown in FIG. 10 in which the tracheal intubation device 142 is comprised of the endotracheal tube adaptor 144, the tubing component 146, the endotracheal tube 148 and the intubation stylet 150 which are the same as the endotracheal tube adaptor 12, the tubing component 14, the endotracheal tube 28 and the intubation stylet 34 of the tracheal intubation device 10 shown in FIG. 1 respectively. The tracheal intubation device 142 uses a control unit 152 that is the same as control unit 72 shown in FIG. 7.

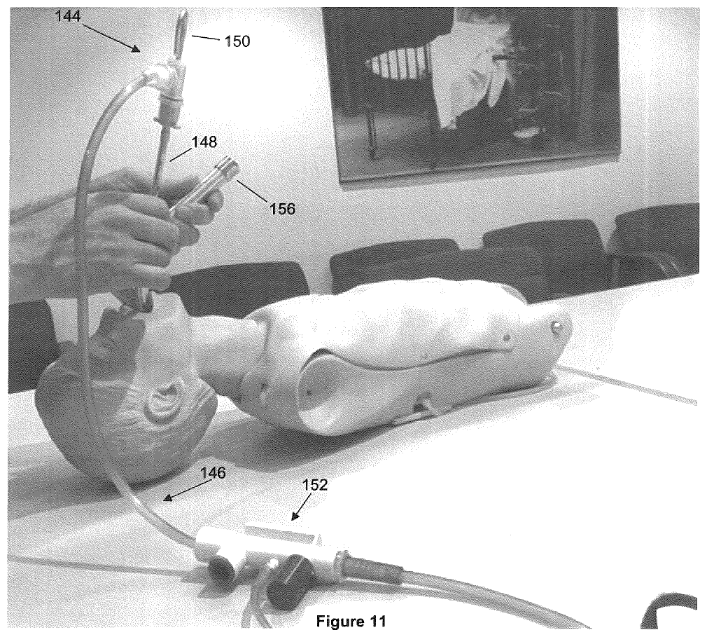

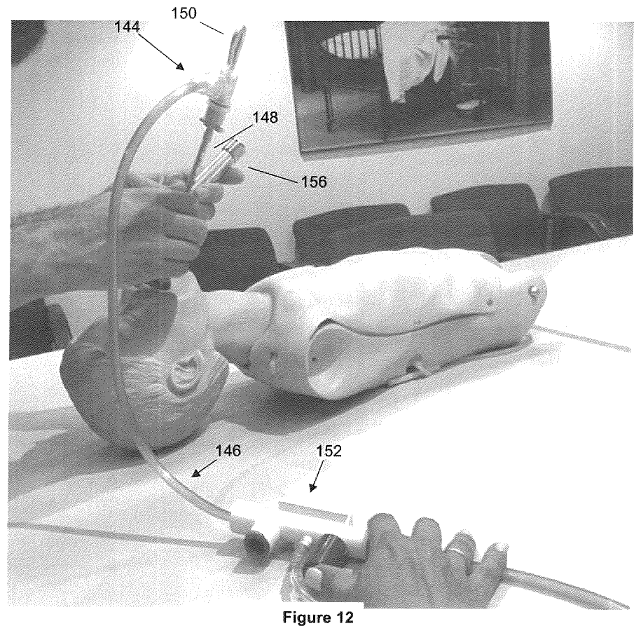

An embodiment of the present disclosure is shown in FIGS. 11 and 12 wherein the tracheal intubation device 142. During intubation, the intubator inserts the intubation stylet 150 into the endotracheal tube 148 through the stylet accommodator 154 of the endotracheal tube adaptor 144 to make the endotracheal tube 148 sufficiently rigid such that the endotracheal tube is able to advance lower in the patient's airway instead of bending as it slides along airway surfaces. During intubation, a laryngoscope 156 is used to generate a view of the airway this occupies one of the intubator's hands. The intubator holds the endotracheal tube with stylet with his or her other hand to manipulate the endotracheal tube toward the patient's vocal cords. Suction may be operated by an assistant who is positioned with the control unit such that the intubator is able to easily manipulate the endotracheal tube, as shown in FIG. 12.

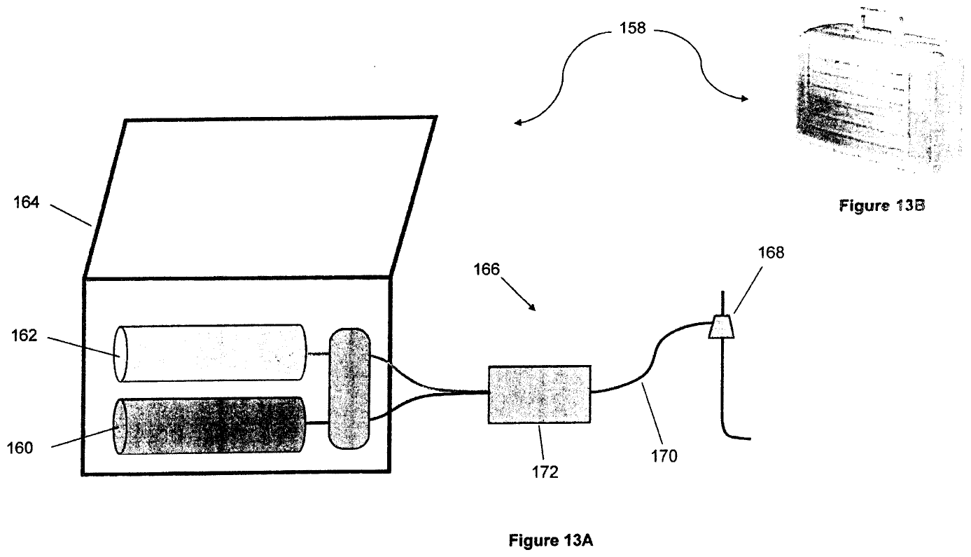

In an alternate embodiment of the disclosure, the tracheal intubation device may be included in a portable unit 158 with stand-alone medical gas and suction supplies, as shown in FIG. 13A. In this embodiment 158, the medical gas supply 160 and suction supply 162 are housed in a portable case 164 with the tracheal intubation device 166 and an optional laryngoscope. This portable unit 158 enables one to perform an intubation in an area where separate medical gas and suction supplies are not otherwise available. In this portable unit 158 the endotracheal tube adaptor 168, tubing component 170 and control unit 172 of tracheal intubation device 166 are the same as endotracheal tube adaptor 12, tubing component 14 and control unit 16 of tracheal intubation device 10 respectively. FIG. 13B shows the portable unit 158 closed and ready for transport.

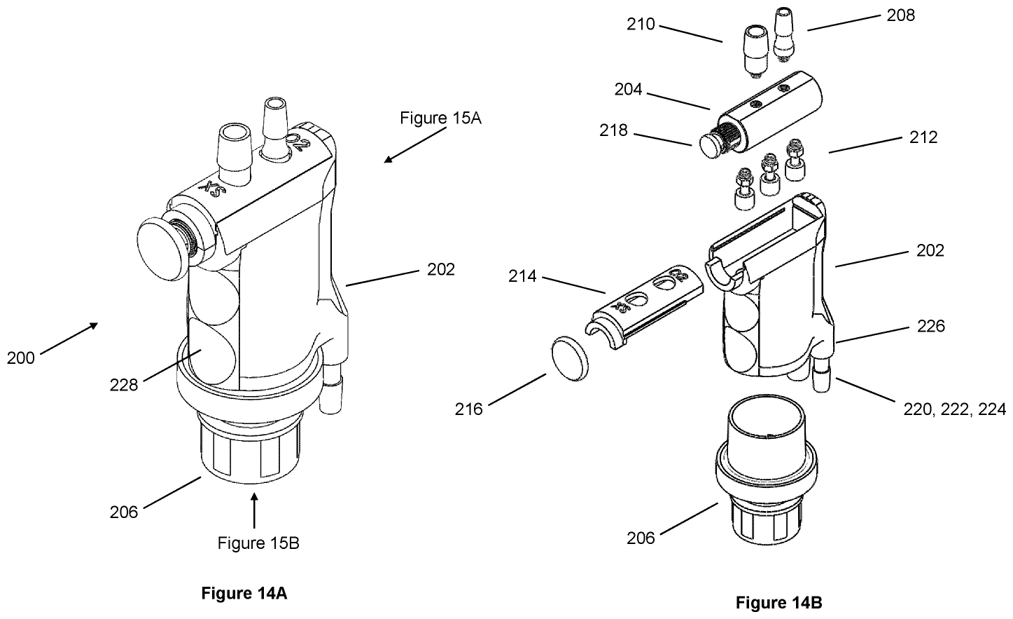

Another embodiment of the system is shown in FIGS. 14A to 20B. A hand operated control unit 200, shown in FIGS. 14A and 14B, generally comprises a body 202, a control apparatus 204, and a pressure limiting device 206 (such as but not limited to an adjustable medical pressure relief valve 206).

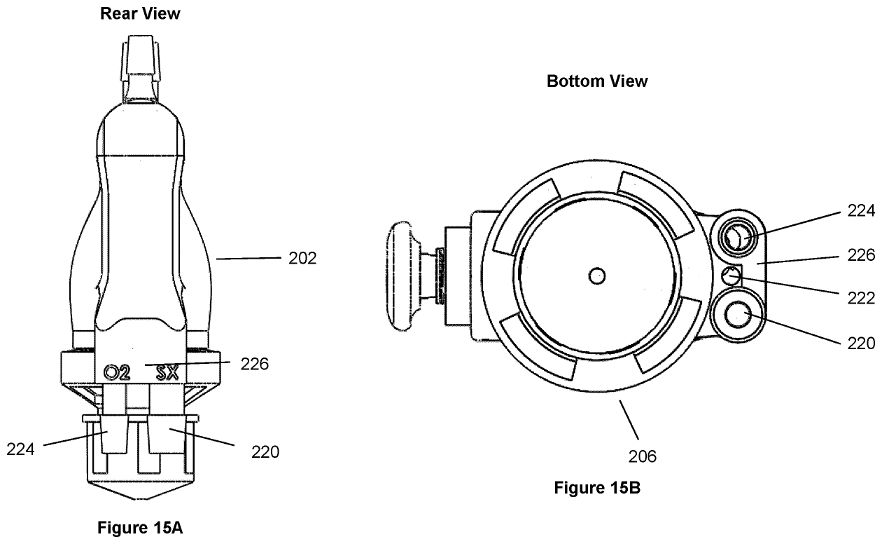

A medical gas input port 208 and suction input port 210 are connected to the control apparatus 204, shown in FIG. 14B. Internal connectors 212 are connected to the control apparatus 204 and press-fit into the body 202. A lid 214 slides into the body 202 to contain the control apparatus 204 and a button cap 216 is press-fit to the spring-loaded button 218 of the control apparatus 204. A pressure limiting device 206 is press-fit into the device body 202. A suction output port 220, exhaust port 222, and medical gas output port 224 are integrated in the body 202 and are best shown in FIGS. 15A and 15B. The exhaust port 222 directs either medical gas or suction to atmosphere, depending on the state of the control apparatus 204. The exhaust port is protected from an operator with an extension 226 of the body 202, best shown in FIGS. 15A and 15B. Finger grip cutouts 228 enable comfortable grasping of the device body 202 by an operator. The suction output port 220, exhaust port 222, and medical gas output port 224 are located toward the rear and bottom of the device body 202 for the same purpose.

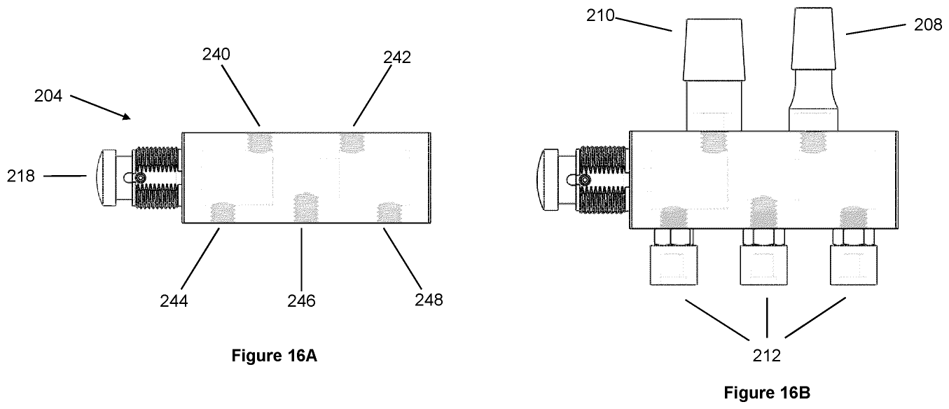

The control apparatus 204 is an off-the-shelf spring-return 5-way valve with two input ports 240 and 242, and three output ports 244, 246, and 248, shown in FIG. 16A. In the default state, or non-activated state, the input port 240 is directed to output port 246, and input port 242 is directed to output port 248. The output port 244 is idle. In the activated state, input port 240 is directed to output port 244, and input port 242 is directed to output port 246. The output port 248 is idle.

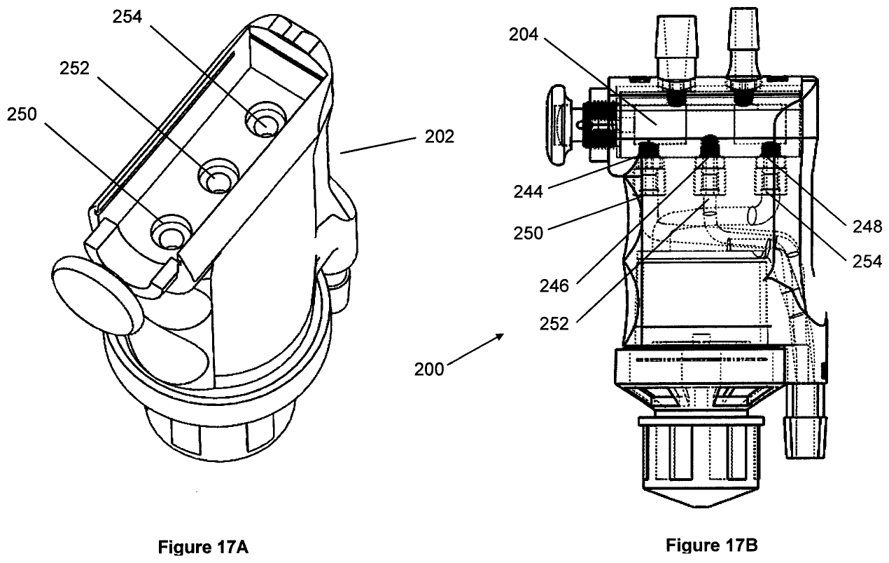

Shown in FIG. 16B is a medical gas input port 208 is attached to input port 242, and a suction input port 210 is attached to input port 240 on the control apparatus 204. Internal pipe connectors 212 are connected to output ports 244, 246, and 248 on the control apparatus 204. The control apparatus 204 assembled with internal pipe connectors 212 is press-fit into the body 202, with an airtight seal formed between the internal pipe connectors 212 and internal body channels 250, 252, and 254 shown in FIG. 17A.

Control apparatus 204 output port 244 is connected to internal body channel 250 via an internal pipe connector 212, control apparatus 204 output port 246 is connected to internal body channel 252 via an internal pipe connector 212, and control apparatus 204 output port 248 is connected to internal body channel 254 via an internal pipe connector 212, as shown in FIG. 17B. The internal body channels 250, 252, and 254 are integrated into the body of the device 202 and direct medical gas and suction flow.

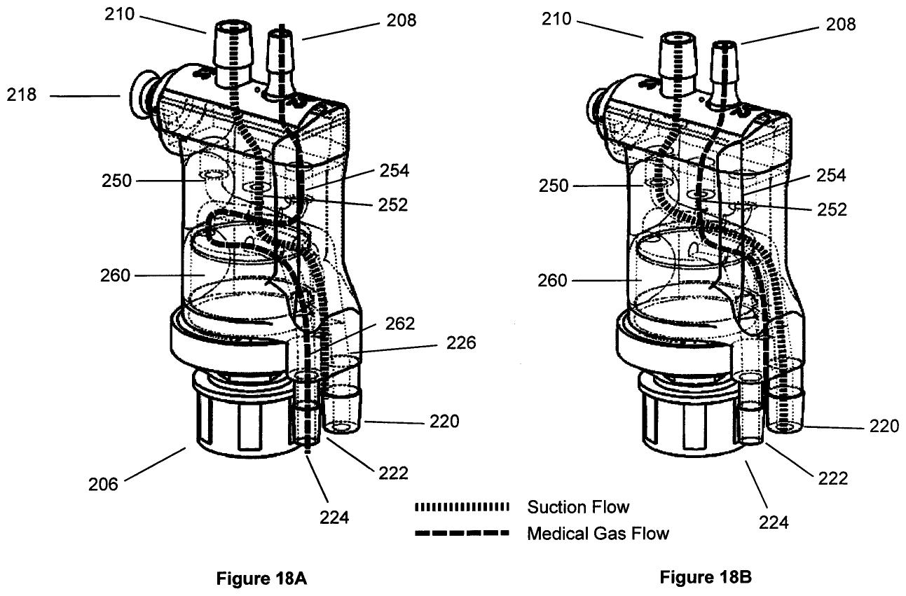

FIGS. 18A and 18B show the medical gas and suction flow channels internally in the device body. Separate, dedicated suction and medical gas channels within the control unit 200 allow for the connection to separate, dedicated tubing for medical gas and suction lines. Compared to a single channel for both medical gas and suction, such as the embodiment shown in FIG. 1, this configuration avoids the requirement for suctioned material to pass through the device before medical gas is supplied to the patient; medical gas can be delivered immediately once suction is no longer required.

The control apparatus 204 enables an operator to switch from medical gas supply to on-demand suction. The device is configured to deliver medical gas when in the non-activated state. As shown in FIG. 18A, when the control apparatus 204 is not activated, a medical gas source (not shown) is connected to medical gas input port 208 and flows via dedicated medical gas channel 254 inside the device body 202. The medical gas enters the main chamber 260 which is directly connected to the pressure limiting device 206 via press-fit. The medical gas exits the main chamber 260 via a medical gas channel 262 to the output port 224. If the local pressure inside the chamber 260 exceeds the current setting of the pressure limiting device 206, excessive pressure will vent to the atmosphere via a spring-loaded vent mechanism located in the pressure limiting device 206. In this non-activated state, a suction source (not shown) is connected to the suction input port 210 and suction is diverted to the exhaust channel 252 and to atmosphere via exhaust port 222.

The body extension 226 protects an operator from unintentionally preventing suction flow from entering the exhaust port 222. The dedicated suction channel 250 and suction output port 220 are idle in this non-activated configuration, and only medical gas is delivered to the patient.

In the activated configuration shown in FIG. 18B, the control apparatus 204 is activated via the spring-loaded control unit button 218. The activated configuration alters the internal configuration of the control apparatus 204 and diverts medical gas from the medical gas input port 208 to the exhaust channel 252, where it is vented to atmosphere at exhaust port 222. The extension 226 protects the operator from unintentionally blocking the medical gas venting from exhaust port 222. The suction force enters the device at suction input port 210 and flows through dedicated suction channel 250 inside the device body 202 and continues to the suction output port 220. In a separate embodiment, the medical gas could be limited to a preselected flow rate to avoid reducing the effective suction force flowing through the endotracheal tube, without venting medical gas to atmosphere.

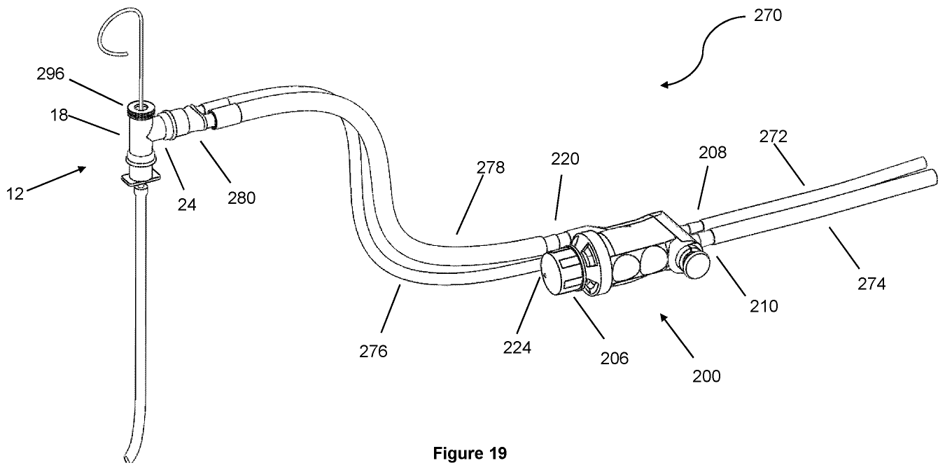

An embodiment of the present disclosure is shown in FIG. 19 at 270, in which the endotracheal tube adapter 12, the same as described by FIG. 1, is included with a control unit 200 and tubing 272, 274, 276, and 278. Standard medical gas tubing 272 conducts medical gas flow between the source (not shown) and the control unit 200. The medical gas tubing 272 is connected to the control unit 200 via medical gas input port 208. Standard suction tubing 274 conducts suction between the source (not shown) and the control unit 200. The suction tubing 274 is connected to the control unit 200 via suction input port 210. Standard medical gas tubing 276 conducts medical gas between the control unit 200 medical gas output 224 and flow Y-junction 280. Suction tubing 278 conducts suction between the control unit 200 suction output port 220 and flow Y-junction 280.

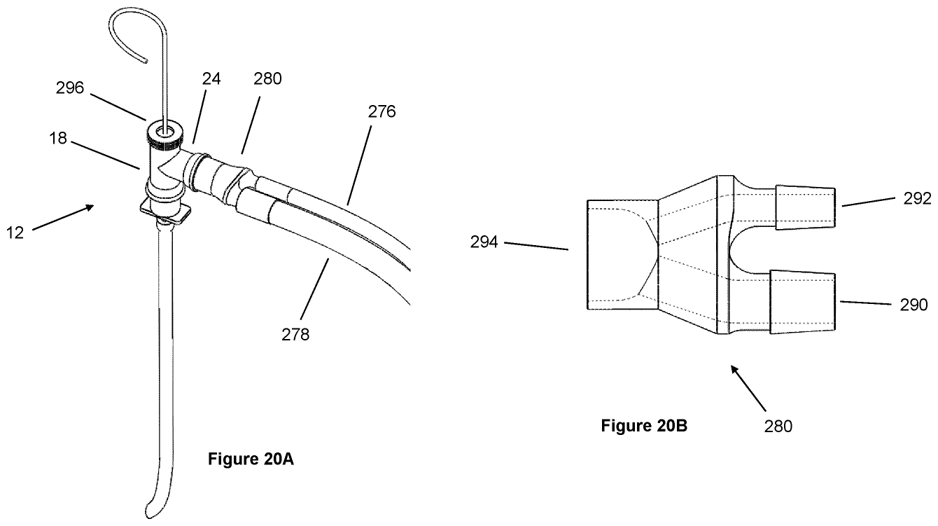

Shown in FIGS. 20A and 20B, the flow Y-junction 280 connects via press-fit of the flow Y-junction output 294 to the tubing port 24 of the body 18 of the endotracheal tube adapter 12. The endotracheal tube adapter 12 functions as described in FIG. 1. A standard medical gas connector 292 and standard suction connector 290 are integrated in the flow Y-splitter 280, best shown in FIG. 20B. Suction tubing 278 attaches at one end to the suction connector 290 of the flow Y-junction 280 attached to the endotracheal tube adaptor 12 and attaches at the other end to the suction output 220 of the control unit 200. The oxygen tubing 276 attaches at one end to the medical gas connector 292 of the flow Y-junction 280 attached to the endotracheal tube adaptor 12. The flow Y-junction 280 converts the single channel of the endotracheal tube adapter 12 to dedicated medical gas and suction channels.

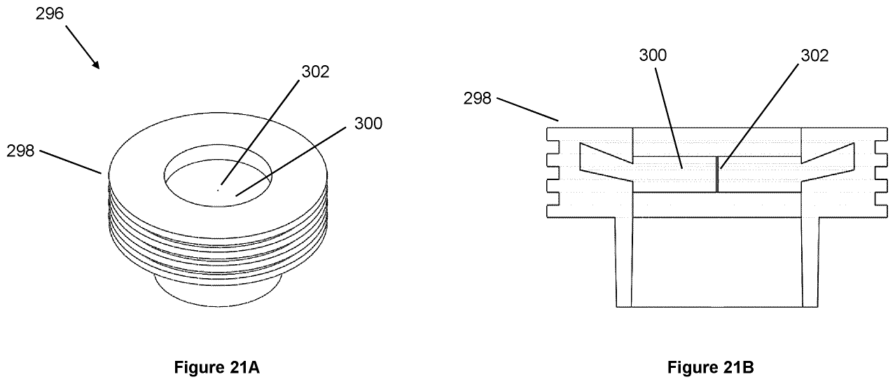

Shown in FIG. 20A, a sealing cap 296 is press-fit into the top portion of the body 18 of the endotracheal tube adapter 12. The sealing cap is comprised of a plastic housing 298 with an embedded silicon gasket 300, best shown in FIGS. 21A and 21B. A stylet insertion opening 302 is a small puncture in the center of the silicon gasket 300 and provides an opening for the stylet 34 to be inserted into the endotracheal tube adapter 12. The sealing cap 296 prevents any medical gas or suction leakage from the endotracheal adapter 12 during the insertion of the stylet 34, and seals completely to prevent medical gas or suction leakage when the stylet is fully removed.

Ventilation includes both conventional and non-conventional modalities. Non-conventional modalities including but not limited to high frequency oscillation (HFO) in which delivered medical gas is rapidly moved back and forth to provide active inspiration and active expiration. The oscillation is achieved using mechanical methods such as a piston, membrane, flow interrupter, or switching valves. Non-conventional modalities also include high or low frequency jet ventilation, in which high pressured medical gas is intermittently delivered by means of flow interruption. High frequency jet ventilation achieves flow interruption by mechanical methods such as solenoid valves, fluidic or rotating valves, and other pneumatically or electronically controlled devices. Low frequency jet ventilation is typically achieved by hand-triggered flow interruption devices. The control unit of the present airway device for delivering medical gas to a patient can be readily modified to operate in these non-conventional modalities. For example, the present control unit may be modified as disclosed above to deliver gas in any of these non-conventional modalities.

In an embodiment the present disclosure provides an airway access device configured to be coupled to a patient's airway and which comprises;
an airway access device adaptor connected to and in flow communication with the airway access device;
a medical gas input control unit including a housing having first and second ends, the second end of said control united being connected to the airway access device adapter, the control unit including a medical gas supply coupling located at the first end of the housing connectable to a source of medical gas, a pressure limiting device located downstream of the medical gas supply coupling, and wherein the pressure limiting device is configured to limit medical gas flow upon a pressure of medical gas in the pressure limiting device exceeding a preselected threshold pressure; and
the airway access device adapter is configured to form a gas tight seal with the airway access device when coupled to said airway access device.

In an embodiment the device medical gas input control unit further comprises a suction supply coupling located at the first end of the housing and being connectable to a source of suction, and the control unit may include a hand-operated control mechanism for controlling both suction and medical gas flow.

In an embodiment the hand-operated control mechanism is a finger activated switch for switching between the source of suction and the source of medical gas, and the control unit may be configured such that when the finger activated mechanism is not activated, medical gas flows to the airway access device, and when activated the medical gas flow is stopped and suction is applied to the airway access device, so that medical gas and suction cannot be provided at the same time.

In an embodiment the hand-operated control mechanism is a finger activated switch for switching between the source of suction and the source of medical gas and the control unit may be configured such that when the finger activated mechanism is not activated, medical gas flows to the airway access device, and when activated the medical gas flow is reduced to a preselected flow rate and suction is simultaneously applied to the airway access device, so that medical gas and suction are provided at the same time.

In an embodiment the airway access device adaptor is connected to, and in flow communication with, the airway access device by means of elongate flexible tubing.

In an embodiment the pressure limiting device is any one of a pressure relief valve, a pressure regulating valve, a rupture disc, an aperture, or a breather vent.

In an embodiment the airway access device is any one of an endotracheal tube, Laryngeal mask airway, tracheostomy tube, nasopharyngeal airway or tube, oropharyngeal tube, cricothyrotomy tube, and the like.

In an embodiment the present disclosure also provides an airway device for delivering medical gas to a patient, comprising:
an airway access device configured to be coupled to a patient's airway;
an airway access device adaptor connected to and in flow communication with the airway access device;
a medical gas input control unit including a housing having first and second ends, the second end of said control united being connected to said airway access device adapter, the control unit including a medical gas supply coupling located at said first end of the housing connectable to a source of medical gas, a pressure limiting device located downstream of the medical gas supply coupling, and wherein the pressure limiting device is configured to limit medical gas flow upon a pressure of medical gas in the pressure limiting device exceeding a preselected threshold pressure;
a suction supply coupling located at the first end of the housing and being connectable to a source of suction, the control unit including a hand-operated control mechanism for controlling both suction and medical gas flow; and
the airway access device adapter being configured to form a gas tight seal with the airway access device when coupled to the airway access device.

In an embodiment the hand-operated control mechanism is a finger activated switch for switching between the source of suction and the source of medical gas, the control unit being configured such that when the finger activated mechanism is not activated, medical gas flows to the airway access device, and when activated the medical gas flow is stopped and suction is applied to the airway access device, so that medical gas and suction cannot be provided at the same time.

In an embodiment the hand-operated control mechanism is a finger activated switch for switching between the source of suction and the source of medical gas, the control unit being configured such that when the finger activated mechanism is not activated, medical gas flows to the airway access device, and when activated the medical gas flow is reduced to a preselected flow rate and suction is simultaneously applied to the airway access device, so that medical gas and suction are provided at the same time.

In an embodiment the airway access device adaptor is connected to, and in flow communication with, the airway access device by means of elongate flexible tubing.

In an embodiment the pressure limiting device is any one of a pressure relief valve, a pressure regulating valve, a rupture disc, an aperture, or a breather vent.

In an embodiment the airway access device is any one of an endotracheal tube, Laryngeal mask airway, tracheostomy tube, nasopharyngeal airway or tube, oropharyngeal tube, cricothyrotomy tube, and the like.

In an embodiment the present disclosure provides an airway device for delivering medical gas to a patient, comprising:
an endotracheal tube having a first end configured to be inserted into a patient's airway and an opposed second end;
an endotracheal tube adaptor attached to the second end configured to have one end of an elongate flexible tube attached thereto; and
a hand operated medical gas input control unit including a housing having first and second opposed ends, a second end of the elongate flexible tube being connected to the second end of said housing, the control unit including a medical gas supply coupling connectable to a source of medical gas and a pressure limiting device located downstream of the medical gas supply coupling between said medical gas supply coupling and the first end of the housing, wherein the pressure limiting device is configured to vent the medical gas out of the housing upon a pressure of medical gas in the flexible tube exceeding a preselected threshold pressure and
the endotracheal tube adapter configured to form a gas tight seal with said endotracheal tube when coupled to said airway access device.

In this embodiment the medical gas input control unit further comprises a suction supply coupling located at the first end of the housing and being connectable to a source of suction, the control unit including a hand-operated control mechanism for controlling both suction and medical gas flow.

In this embodiment the pressure limiting device is a pressure relief valve is located adjacent to the second end of the housing with the medical gas supply coupling being located between said pressure relief valve and said suction supply coupling.

In this embodiment the suction supply coupling is located adjacent to the first end of the housing, and wherein the medical gas supply coupling is located adjacent to the second opposed end, and wherein said pressure limiting device is a pressure relief valve located between the medical gas supply coupling and the suction supply coupling.

In summary, the present disclosure provides an airway device for delivering medical gas to a patient and suction, ideally but not exclusively intended for the delivery of apneic oxygenation (or other medical gas), with a pressure relief valve for protection from high pressure. The device includes an intubation stylet and optional medical aspiration directly through an endotracheal patient tube during intubation, without need for a bendable soft catheter. The patient end allows for connection to a standard endotracheal tube connector and the devices includes a self-sealing port at the patient end which allows for the application of an intubation stylet while maintaining airflow with minimal leakage. The patient end of the device and the control unit for controlling medical gas flow and suction are separated by semi-rigid or flexible tubing. The control unit includes a port for the application of standard small bore medical gas tubing that allows for the delivery of apneic oxygenation during insertion of the endotracheal tube into the patient's airway. The control unit includes a pressure release valve that protects the patient airway from high pressures. The control unit further includes a plunger switch that can be engaged by the clinician to provide access medical aspiration directly to the endotracheal tube. The control unit also includes a port that can be connected to standard suction tubing to regulated suction.

It will be understood that while the present device has been described and illustrated as being configured for providing oxygen during the apneic portion of intubation with an endotracheal tube, this device may have other applications. It is essentially a device that allows and controls flow of medical suction and medical gas (that may or may not be oxygen) to an airway device (that may or may not be an endotracheal tube).

The foregoing description of the preferred embodiments of the disclosure has been presented to illustrate the principles of the disclosure and not to limit the disclosure to the particular embodiment illustrated. It is intended that the scope of the disclosure be defined by all of the embodiments encompassed within the following claims and their equivalents.

1.-17. (canceled)

18. An airway device for delivering medical gas to a patient, comprising:
an airway access device having a first end configured to be operably coupled to a patient's airway;
an airway access device adaptor having an adaptor body connected to an opposed second end of the airway access device along a first end of the adaptor body and connected to an elongate flexible tube component along a tubing port of the adaptor body and a first end of the elongate flexible tube component, the airway access device adaptor in flow communication with the airway access device;
a medical gas input control unit including a housing having first and second ends, said second end of said housing being connected to a second end of the elongate flexible tube component, said control unit including a medical gas supply coupling located at said first end of said housing connectable to a source of medical gas, a pressure limiting device located downstream of said medical gas supply coupling, and wherein said pressure limiting device is configured to be able to select a threshold pressure in a preselected pressure range at which medical gas is vented in order to limit medical gas flow upon a pressure of medical gas in the pressure limiting device exceeding a selected threshold pressure;
said airway access device adaptor configured to form a gas tight seal with said endotracheal tube when coupled to said endotracheal tube; and
wherein in operation, once the first end of the endotracheal tube is inserted into the patient's airway, a clinician operating the airway device can move the control unit independent of the airway access device adaptor and endotracheal tube to provide the clinician a clear and unobstructed view of the patient's airway.

19. The device according to claim 18 wherein said medical gas input control unit further comprises a suction supply coupling located at said first end of said housing and being connectable to a source of suction, said control unit including a hand-operated control mechanism for controlling both suction and medical gas flow.

20. The device according to claim 19 wherein said hand-operated control mechanism is a finger activated switch for switching between the source of suction and the source of medical gas, said control unit being configured such that when said finger activated mechanism is not activated, medical gas flows to the airway access device, and when activated the medical gas flow is stopped and suction is applied to the airway access device, so that medical gas and suction cannot be provided at the same time.

21. The device according to claim 19 wherein said hand-operated control mechanism is a finger activated switch for switching between the source of suction and the source of medical gas, said control unit being configured such that when said finger activated mechanism is not activated, medical gas flows to the airway access device, and when activated the medical gas flow is reduced to a preselected flow rate and suction is simultaneously applied to the airway access device, so that medical gas and suction are provided at the same time.

22. The device according to claim 18, wherein said pressure limiting device is any one of a pressure relief valve, a pressure regulating valve, a rupture disc, an aperture, or a breather vent.

23. The device according to claim 18, wherein said airway access device adaptor is connected to, and in flow communication with, said airway access device by means of elongate flexible tubing.

24. The device according to claim 18, wherein said airway access device is any one of an endotracheal tube, laryngeal mask airway, tracheostomy tube, nasopharyngeal airway or tube, oropharyngeal tube or a cricothyrotomy tube.

25. An airway device for delivering medical gas to a patient, comprising:
an airway access device having a first end configured to be operably coupled to a patient's airway;
an airway access device adaptor having an adaptor body connected to an opposed second end of the airway access device along a first end of the adaptor body and connected to an elongate flexible tube component along a tubing port of the adaptor body and a first end of the elongate flexible tube component, the airway access device adaptor in flow communication with the airway access device;
a medical gas input control unit including a housing having first and second ends, said second end of said housing being connected to a second end of the elongate flexible tube component, said control unit including a medical gas supply coupling located at said first end of said housing connectable to a source of medical gas, a pressure limiting device located downstream of said medical gas supply coupling, and wherein said pressure limiting device is configured to limit medical gas flow upon a pressure of medical gas in the pressure limiting device exceeding a preselected threshold pressure;
a suction supply coupling located at said first end of said housing and being connectable to a source of suction, said control unit including a hand-operated control mechanism for controlling both suction and medical gas flow;
said airway access device adaptor configured to form a gas tight seal with said airway access device when coupled to said airway access device; and
wherein in operation, once the first end of the airway access device is operably coupled to the patient's airway, a clinician operating the airway device can move the control unit independent of the airway access device adaptor and airway access device to provide the clinician a clear and unobstructed view of the patient's airway.

26. The device according to claim 25 wherein said hand-operated control mechanism is a finger activated switch for switching between the source of suction and the source of medical gas, said control unit being configured such that when said finger activated mechanism is not activated, medical gas flows to the airway access device, and when activated the medical gas flow is stopped and suction is applied to the airway access device, so that medical gas and suction cannot be provided at the same time.

27. The device according to claim 25 wherein said hand-operated control mechanism is a finger activated switch for switching between the source of suction and the source of medical gas, said control unit being configured such that when said finger activated mechanism is not activated, medical gas flows to the airway access device, and when activated the medical gas flow is reduced to a preselected flow rate and suction is simultaneously applied to the airway access device, so that medical gas and suction are provided at the same time.

28. The device according to claim 25, wherein said pressure limiting device is any one of a pressure relief valve, a pressure regulating valve, a rupture disc, an aperture or a breather vent.

29. The device according to claim 25, wherein said airway access device adaptor is connected to, and in flow communication with, said airway access device by means of elongate flexible tubing.

30. The device according to claim 25, wherein said airway access device is any one of an endotracheal tube, laryngeal mask airway, tracheostomy tube, nasopharyngeal airway or tube, oropharyngeal tube or a cricothyrotomy tube.

31. An airway device for delivering medical gas to a patient, comprising:
an endotracheal tube having a first end configured to be operably inserted into a patient's airway and an opposed second end;
an endotracheal tube adaptor having an adaptor body connected to the opposed second end along a first end of the adaptor body and connected to a first end of an elongate flexible tube component along a tube port of the adaptor body, the airway access device adaptor in flow communication with the endotracheal tube;
a hand operated medical gas input control unit including a housing having first and second opposed ends, a second end of said elongate flexible tube component being connected to said second end of said housing, said control unit including a medical gas supply coupling connectable to a source of medical gas and a pressure limiting device located downstream of said medical gas supply coupling between said medical gas supply coupling and said first end of said housing, wherein said pressure limiting device is configured to vent the medical gas out of said housing upon a pressure of medical gas in the flexible tube exceeding a preselected threshold pressure;
said endotracheal tube adaptor configured to form a gas tight seal with said endotracheal tube when coupled to said airway access device; and
wherein in operation, once the first end of the endotracheal tube is inserted into the patient's airway, a clinician operating the airway device can move the hand operated medical gas control unit independent of the airway access device adaptor and endotracheal tube to provide the clinician a clear and unobstructed view of the patient's airway.

32. The device according to claim 31, wherein said hand operated medical gas input control unit further comprises a suction supply coupling coupled to said first end of said housing and being connectable to a source of suction, said hand operated medical gas input control unit including a hand-operated control mechanism for controlling both suction and medical gas flow.

33. The device according to claim 32, wherein said pressure limiting device is a pressure relief valve located adjacent to said second end of said housing with the medical gas supply coupling being located between said pressure relief valve and said suction supply coupling.

34. The device according to claim 32 wherein said medical gas supply coupling is coupled to said second opposed end, and wherein said pressure limiting device is a pressure relief valve located between said medical gas supply coupling and said suction supply coupling.

35. The device according to claim 31, wherein said pressure limiting device is any one of a pressure relief valve, a pressure regulating valve, a rupture disc, an aperture or a breather vent.
